(19) United States
(12) Patent Application Publication
(10) Pub. No.: US 2015/0002811 A1
(43) Pub. Date: Jan. 1, 2015

(54) IMAGE PROCESSING APPARATUS AND IMAGE PROCESSING METHOD

(71) Applicant: CANON KABUSHIKI KAISHA, Tokyo (JP)

(72) Inventor: Yuji Ota, Yokohama-shi (JP)

(21) Appl. No.: 14/312,491

(22) Filed: Jun. 23, 2014

(30) Foreign Application Priority Data
Jun. 28, 2013 (JP) PCT/JP2013/067835

(51) Int. Cl. A61B 3/12; A61B 5/00; A61B 3/14

(52) U.S. Cl. CPC A61B 3/12; A61B 3/14; A61B 5/0059
USPC 351/206; 351/246

(57) ABSTRACT

An image processing apparatus includes a determination unit and a changing unit. The determination unit determines a first region and a second region, the first region being a portion of a first fundus image acquired by performing autofluorescence imaging on a fundus of an eye to be inspected, the second region being a portion of a second fundus image acquired by performing autofluorescence imaging on the fundus at a time different from that of the first fundus image and being at a position corresponding to the first region. The changing unit changes a gradation of at least one of the first and second fundus images on the basis of pixel values of the first and second regions.

This application claims the benefit of International Patent Application No. PCT/JP2013/067835, filed Jun. 28, 2013, which is hereby incorporated by reference herein in its entirety.

### TECHNICAL FIELD

The present invention relates to an image processing apparatus and an image processing method for processing images.

### BACKGROUND ART

Recently, fundus autofluorescence imaging (FAF: Fundus Auto-Fluorescence) has been receiving attention. PTL1 has disclosed that, in autofluorescence imaging, the fundus is illuminated with light around 550 nm as excitation light and light around 640 nm is received as autofluorescence caused by lipofuscin. A user may detect age-related macular degeneration or the like at an early stage by checking lipofuscin using a fundus image acquired as a result of this light reception.

### CITATION LIST

### Patent Literature

PTL 1 Japanese Patent Laid-Open No. 2006-247076

Here, when lipofuscin accumulates near a macula, there is a high probability that age-related macular degeneration will occur. Thus, it is important to check changes in lipofuscin that occur over time. Here, even when the amount of light used for imaging is fixed when a plurality of fundus images are captured at different times, since the amount of autofluorescence caused by lipofuscin is small, even a slight change in an illumination state of a fundus may result in a large change in the intensity of a region which is less likely to be affected by changes in lipofuscin that occur over time in the plurality of fundus images. As a result, a user may falsely diagnose a region which is less likely to be affected by changes in lipofuscin that occur over time in a fundus image as a region where a change in lipofuscin occurs.

An object of the present invention is to reduce false diagnoses in which a user diagnoses a region which is less likely to be affected by changes in lipofuscin that occur over time in a fundus image as a region where a change in lipofuscin occurs.

### SUMMARY OF INVENTION

An image processing apparatus according to the present invention includes determination means that determines a first region and a second region, the first region being a portion of a first fundus image acquired by performing autofluorescence imaging on a fundus of an eye to be inspected, the second region being a portion of a second fundus image acquired by performing autofluorescence imaging on the fundus at a time different from that of the first fundus image and being at a position corresponding to the first region, and changing means that changes a gradation of at least one of the first and second fundus images on the basis of pixel values of the first and second regions.

Further features of the present invention will become apparent from the following description of exemplary embodiments with reference to the attached drawings.

### BRIEF DESCRIPTION OF DRAWINGS

FIG. 1 is a schematic diagram illustrating an example of an entire configuration of a first embodiment.

FIGS. 2A and 2B are diagrams associated with alignment and focusing of the first embodiment.

FIG. 3 is a flowchart of an image-gradation changing process of the first embodiment.

FIG. 4 is a block diagram of the image-gradation changing process of the first embodiment.

FIGS. 5A to 5E are diagrams illustrating pixel brightness value versus image height of the first embodiment.

FIGS. 6A and 6B are diagrams associated with shading correction of a pupil diameter of the first embodiment.

FIG. 7 includes diagrams associated with an extraction unit of the first embodiment.

FIGS. 8A to 8D are diagrams illustrating image brightness before and after gradation changing of the first embodiment.

FIG. 9 is a diagram associated with a time when images are selected in a second embodiment.

FIG. 10 is a diagram associated with a time when images are selected in the second embodiment.

FIG. 11 is a flowchart of image information display of the second embodiment.

FIG. 12 is a block diagram of the image information display of the second embodiment.

FIG. 13 is a diagram illustrating ROI selection of the second embodiment.

FIG. 14 includes diagrams associated with time-series change display of the second embodiment.

### DESCRIPTION OF EMBODIMENTS

An image processing apparatus according to a present embodiment includes, first, determination means that determines a first region, which is a portion of a first fundus image (an example of a first image) acquired by performing autofluorescence imaging on the fundus of an eye to be inspected. In addition, the determination means determines a second region, which is a portion of a second fundus image (an example of a second image) acquired at a time different from that of the first fundus image by performing autofluorescence imaging on the fundus and is at a position corresponding to the first region. In addition, the image processing apparatus according to the present embodiment includes changing means that changes the gradation of at least one of the first and second fundus images on the basis of pixel values of the first and second regions (a brightness distribution or the like, which is an example of feature values). Note that, preferably, the determination means determines, as the first and second regions, regions which are less likely to be affected by changes in lipofuscin that occur over time in the first and second fundus images, for example, regions including a blood vessel of the fundus.

That is, the image processing apparatus according to the present embodiment changes the gradation of at least one of the first and second fundus images acquired at different times by performing autofluorescence imaging on the fundus of the eye to be inspected, on the basis of pixel values of the first and second regions that are portions of the first and second fundus images and located at positions corresponding to each other.

Here, in a plurality of fundus images acquired at different times by performing autofluorescence imaging on the fundus of the eye to be inspected, for example, gradations of the plurality of fundus images may be matched using regions which are relatively less likely to be affected by changes in lipofuscin that occur over time such as blood vessels. As a result, in a plurality of fundus images acquired at different times by performing autofluorescence imaging on the fundus of the eye to be inspected, for example, gradations of regions may be matched which are less likely to be affected by changes in lipofuscin that occur over time. Thus, false diagnoses may be reduced in which a user diagnoses a region which is less likely to be affected by changes in lipofuscin in a fundus image as a region where a change in lipofuscin occurs.

Here, lipofuscin is likely to be metabolized near blood vessels but is likely to accumulate near a macula where there are few blood vessels. Thus, changes in lipofuscin that occur over time are relatively small near blood vessels including portions behind the blood vessels. In addition, since blood vessels are concentrated near the optic disk, changes in lipofuscin that occur over time are also relatively small near the optic disk. Thus, preferably, the above-described determination means determines images including a blood vessel of the fundus or the optic disk as the first and second regions.

Note that, this is applicable to objects to be inspected other than the fundus of an eye to be inspected. That is, as a result of, for example, matching gradations of regions which are less likely to affected by changes in lipofuscin that occur over time in images captured at different times, false diagnoses has only to be reduced in which a user diagnoses a region which is less likely to be affected by changes in lipofuscin that occur over time as a region where a change in lipofuscin occurs.

Here, it is desirable that the image processing apparatus according to the present embodiment further include coefficient acquisition means that acquires a coefficient with which pixel values of at least one of the first and second fundus images are to be corrected on the basis of pixel values of the first and second regions. In addition, it is desirable that the image processing apparatus according to the present embodiment further include correction means that corrects pixel values of at least one of the first and second fundus images using the coefficient. As a result, the above-described changing means may change the gradation of at least one of the first and second fundus images on the basis of corrected pixel values. In addition, it is preferable that the above-described changing means change the gradation of the first fundus image such that pixel values of the first region become equal to pixel values of the second region. In addition, it is preferable that the changing means change the gradations of the first and second fundus images such that pixel values of the first and second regions become equal to certain pixel values. The above-described changing means has only to change the gradation of at least one of the first and second fundus images such that pixel values of the first region become almost equal to pixel values of the second region.

In addition, the above-described changing means may also change a feature value of at least one of the first and second images on the basis of feature values of the first and second regions. Here, the above-described determination means may also determine a gradation changing characteristic on the basis of brightness distributions of the first and second regions (an example of the feature values). As a result, the above-described changing means may change the brightness distribution of at least one of the first and second images using the determined gradation changing characteristic.

In the following, embodiments of the present invention will be described with reference to drawings.

### First Embodiment

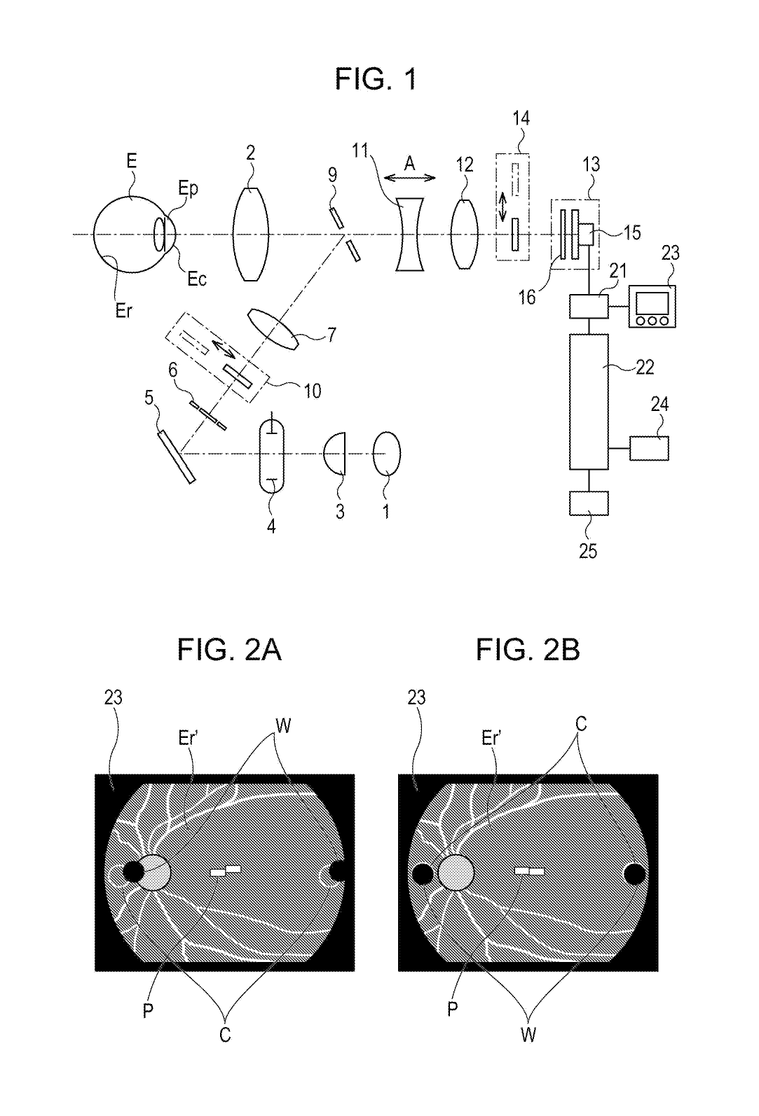

FIG. 1 is a diagram of the configuration of an ophthalmic imaging apparatus in the present embodiment. As the ophthalmic imaging apparatus in the present embodiment, a fundus camera that captures images of the fundus using a two-dimensional sensor or the like, an OCT apparatus or an SLO apparatus that includes scanning means that scans the fundus with light, or the like is preferably used. Note that, the ophthalmic imaging apparatus in the present embodiment may be any imaging apparatus that captures images of an eye to be inspected. Alternatively, in the case of capturing images of an object to be inspected other than an eye to be inspected, the ophthalmic imaging apparatus in the present embodiment may be any imaging apparatus for medical use such as an endoscope.

First, in the ophthalmic imaging apparatus in the present embodiment, a condenser lens 3, an imaging-use light source 4, a mirror 5, an aperture 6 having a ring-shaped opening, a relay lens 7, and a mirror 9, which has a hole, are sequentially arranged along an optical path from an observation-use light source 1 to an object lens 2 positioned in front of an eye E to be inspected. Furthermore, an autofluorescence exciter filter 10, which is an example of wavelength selection means, is arranged between the aperture 6 and the relay lens 7 such that the autofluorescence exciter filter 10 is insertable into and removable from an optical path between the aperture 6 and the relay lens 7. In this manner, an illumination optical system is configured. Note that the autofluorescence exciter filter 10 allows light of a wavelength range from, for example, about 475 nm to about 615 nm, more preferably, a wavelength range from about 530 nm to about 580 nm to pass therethrough. In addition, preferably, the autofluorescence exciter filter 10 blocks light of wavelengths outside this wavelength range. Here, the autofluorescence exciter filter 10 is inserted into the optical path of the illumination optical system when autofluorescence observation imaging is to be performed (in the case where an autofluorescence imaging mode has been selected from among a plurality of imaging modes by use of selection means, which is not illustrated). In addition, the autofluorescence exciter filter 10 is removed from the optical path of the illumination optical system when color imaging is to be performed. Note that, in the case where an SLO apparatus is used, the illumination optical system may be configured such that light of a wavelength for autofluorescence is used by changing a laser light source, the SLO apparatus having a galvanometer mirror or a resonance scanner, the galvanometer mirror and the resonance scanner being examples of scanning means that performs scanning with measurement light with which the eye E to be inspected is to be illuminated.

In addition, a focus lens 11, an imaging lens 12, and a color imaging unit 13 are arranged along an optical path of the mirror 9, which has a hole, in a light-passing-through direction. In addition, an autofluorescence barrier filter 14 (an example of the wavelength selection means) that blocks autofluorescence excitation light and allows fluorescence to selectively pass therethrough is arranged between the imaging lens 12 and the color imaging unit 13 such that the autofluorescence barrier filter 14 is insertable into and removable from an optical path between the imaging lens 12 and the color imaging unit 13. As a result, an observation imaging optical system is configured. The color imaging unit 13 has an imaging device 15 and a tri-color separation color filter 16. Note that, preferably, the autofluorescence barrier filter 14 allows light of, for example, a wavelength range around 640 nm to pass therethrough and blocks light of wavelengths outside this wavelength range. In particular, preferably, the autofluorescence barrier filter 14 blocks light of wavelengths of excitation light with which lipofuscin is excited (for example, a wavelength range having a range from about 530 nm to about 580 nm). Here, the autofluorescence barrier filter 14 is inserted into the optical path of the observation imaging optical system when autofluorescence imaging is to be performed (in the case where the autofluorescence imaging mode has been selected from among the plurality of imaging modes by use of the selection means, which is not illustrated). In addition, the autofluorescence barrier filter 14 is removed from the optical path of the observation imaging optical system when color imaging is to be performed.

In addition, an output of the imaging device 15 is connected to a system controller 22 via an image signal processing unit 21. In addition, a display 23 is connected to the image signal processing unit 21, an observation image of the eye E to be inspected is displayed, and the eye E to be inspected is observed. Moreover, an image recording unit 24 and an operation switch unit 25 are connected to the system controller 22. As a result, a control system of the entire fundus camera is configured. Note that, in these ophthalmic systems, individual blocks are connected in a wired or wireless manner such that communication is possible.

When color-image imaging is performed, luminous flux emitted from the observation-use light source 1 passes through the condenser lens 3 and the imaging-use light source 4, and is reflected by the mirror 5. Light reflected from the mirror 5 passes through the aperture 6 and the relay lens 7, is reflected by a portion of the mirror 9 that is not a hole, the mirror 9 having a hole, and passes through the object lens 2, and the fundus Er of the eye E to be inspected is illuminated with visible light. Here, the autofluorescence exciter filter 10 has been removed from the illumination optical system.

Light reflected from the fundus Er passes through the object lens 2 and a hole of the mirror 9, which has a hole, passes through the focus lens 11 and the imaging lens 12, and forms an image on the imaging device 15. Here, since the autofluorescence barrier filter 14 has been removed from the observation imaging optical system for the fundus, the light reflected from the fundus Er may be observed as is, as a fundus image, on the display 23.

While watching this fundus image, an examiner performs alignment for the eye E to be inspected by moving the apparatus frontward/backward, leftward/rightward, and upward/downward using alignment indicators and an operation unit, which are not illustrated. Furthermore, the examiner performs focusing by moving the focus lens 11 using indicators for focusing.

FIGS. 2A and 2B illustrate an observation state of a fundus image Er′ on the display 23. FIG. 2A illustrates a state in which alignment and focusing have not been completed yet and in which alignment circles C do not match alignment indicators W and indicators P for focusing do not match either. FIG. 2B illustrates a state in which alignment and focusing have been completed and in which the alignment circles C match the alignment indicators W and the indicators P for focusing are arranged in a straight line.

When the examiner presses an imaging switch of the operation switch unit 25 after completion of alignment for and focusing of the fundus image Er′, the system controller 22 causes the imaging-use light source 4 to emit light. Luminous flux emitted from the imaging-use light source 4 and traveling in a path similar to that for luminous flux of the observation-use light source 1 illuminates the fundus Er, and light reflected from the illuminated fundus Er forms an image on the imaging device 15 similarly as in the case of observation. Image data of the formed fundus image Er′ is saved as a color image in the image recording unit 24 via the image signal processing unit 21 and the system controller 22, and the fundus image Er′ is displayed on the display 23.

When autofluorescence observation is to be performed, the autofluorescence exciter filter 10 is inserted into the illumination optical path. Luminous flux emitted from the observation-use light source 1 passes through the condenser lens 3 and the imaging-use light source 4, and is reflected by the mirror 5. Light reflected from the mirror 5 passes through the aperture 6 and the autofluorescence exciter filter 10, is reflected by a portion of the mirror 9 that is not a hole, the mirror 9 having a hole, and passes through the object lens 2, and the fundus Er is illuminated with visible light.

Light reflected from the illuminated fundus Er passes through a pupil Ep, the object lens 2, and the hole of the mirror 9, which has a hole, passes through the focus lens 11 and the imaging lens 12, and forms an image on the imaging device 15. Here, since the autofluorescence barrier filter 14 has been removed from the observation imaging optical system for the fundus, light reflected from the fundus Er out of light of wavelengths that has passed through the autofluorescence exciter filter 10 may be observed as the fundus image Er′.

While watching this fundus image Er′, similarly to as in the case described using FIGS. 2A and 2B, the examiner performs alignment of the apparatus for the eye E to be inspected using the alignment indicators W and performs focusing the indicators P for focusing.

When the examiner presses the imaging switch of the operation switch unit 25 after completion of alignment for and focusing of the fundus image Er′, the system controller 22 inserts the autofluorescence barrier filter 14 into the observation imaging optical system for the fundus and causes the imaging-use light source 4 to emit light. Luminous flux emitted from the imaging-use light source 4 travels in a path similar to that for luminous flux of the observation-use light source 1, and then the fundus Er is illuminated with light of a wavelength that has passed through the autofluorescence exciter filter 10. Light reflected from the illuminated fundus Er passes through the pupil Ep, the object lens 2, and the hole of the mirror 9, which has a hole, passes through the focus lens 11 and the imaging lens 12, and the autofluorescence barrier filter 14 blocks light of wavelengths that has passed through the autofluorescence exciter filter 10. As a result, only fluorescence of the fundus image Er′ passes and forms an image on the imaging device 15.

The fundus image Er′, which has been formed, is changed into a monochrome image by the image signal processing unit 21 and saved as a monochrome image in the image recording unit 24 via the system controller 22. The fundus image Er′, which is a monochrome image, is displayed on the display 23. Note that the image signal processing unit 21 may have a function through which a signal from the imaging device 15 is not processed and a signal to and from the system controller 22 and a signal to and from the display 23 are only transferred. In this case, the image signal processing unit 21 and the system controller 22 may also be integrally configured as, for example, an image processing apparatus.

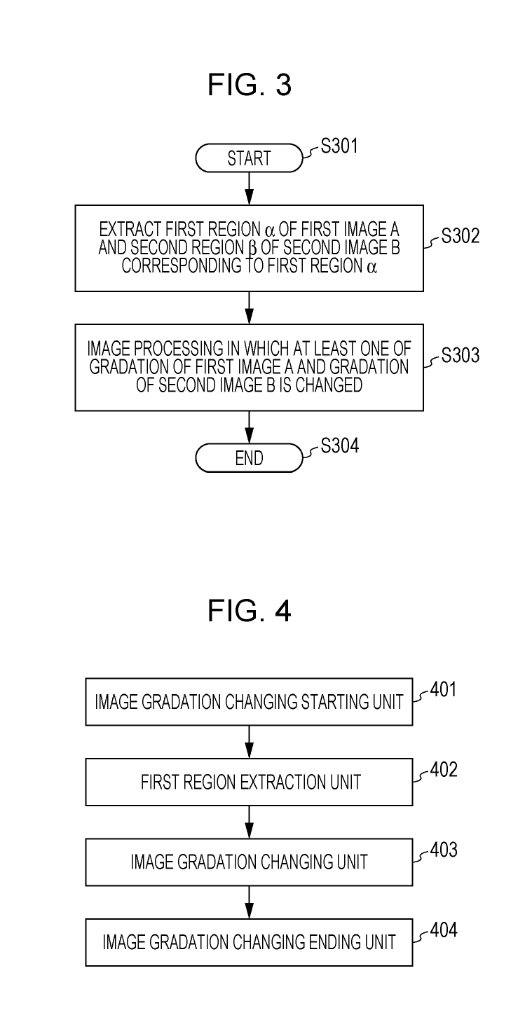

Next, image brightness gradation changing based on brightness information on the first region, which is a characteristic of the present embodiment, will be described using FIGS. 3 to 8D. Here, in the present embodiment, an image gradation changing starting unit 401, a first region extraction unit 402, an image gradation changing unit 403, and an image gradation changing ending unit 404 are functional blocks inside the system controller 22, which is an example of the image processing apparatus. Note that at least one of these functional blocks may be a functional block in the outside such as in the image signal processing unit 21. In addition, these functional blocks are realized using a CPU that the system controller 22 has and a memory in which a program for executing a flowchart to be described below is stored.

FIGS. 3 and 4 are a flowchart and a block diagram for changing the gradation of a captured image, respectively. The image gradation changing starting unit 401 starts a flow for adjusting the gradation of a first image A and the gradation of a second image B (S301), the first image A and the second image B being stored in the image memory 24, the first image A and the second image B being images of the same eye, the second image B being captured on a date different from that of the first image A. Next, the first region extraction unit 402, which is an example of the determination means, extracts a first region α of the first image A and extracts a second region β of the second image B in an interlocked manner, the second region corresponding to the first region α (S302). Here, desirably, the first region α is an image region corresponding to a blood vessel of the fundus image Er′. This is because, in an autofluorescence image, a blood vessel in a blood vessel portion generally absorbs light of wavelengths that has passed through the autofluorescence exciter filter 10 and the blood vessel portion does not show autofluorescence. In addition, a standard position is determined using fundus pattern specifying means in the fundus image Er′, the standard position is set to be an origin, an x axis and a y axis are set by considering rotation and movement of an image, and corresponding regions are regions that have the same x coordinate and the same y coordinate from the origin. Here, the standard position is, for example, an optic disk portion or a blood vessel portion of a fundus image. For example, in the case where a blood vessel portion is set to be a standard position, Japanese Patent Laid-Open No. 5-253215 may be cited as an example.

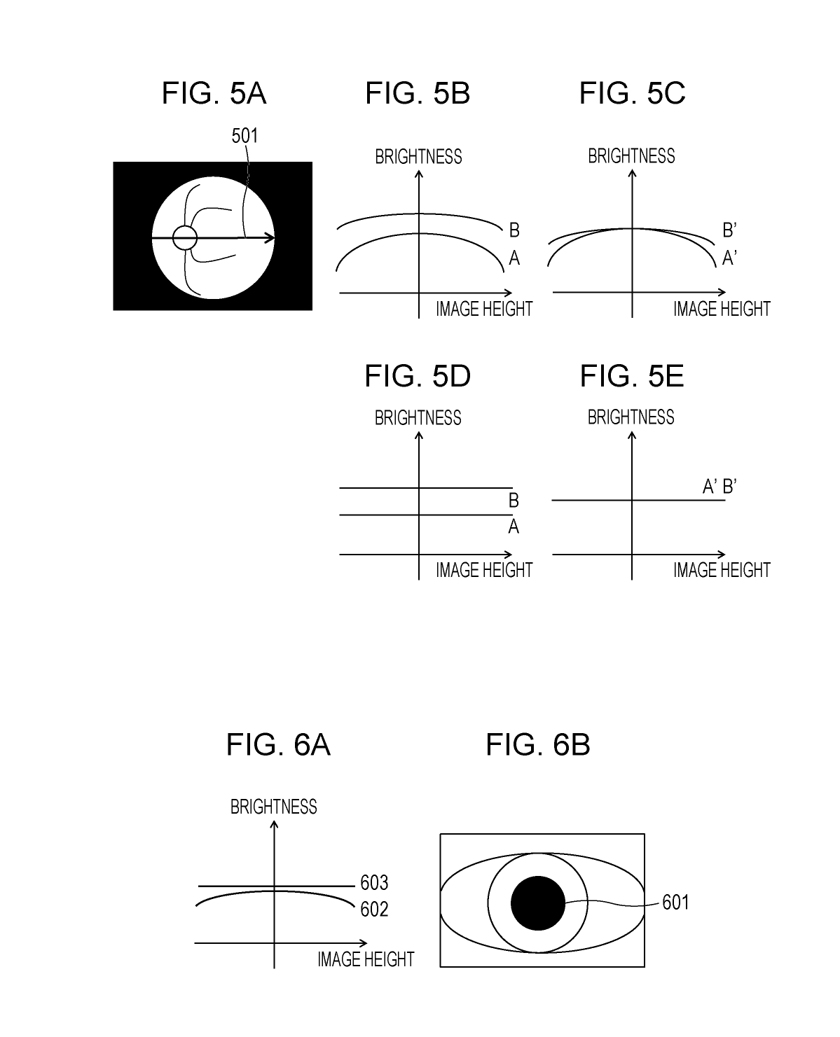

Note that, when imaging is performed in a state in which the pupil Ep of the eye E to be inspected is sufficiently open and sufficient alignment has been achieved, a region different from the first region α may be extracted as the second region β if the fundus image of FIG. 5A is, for example, unlike in FIG. 5B, an image whose brightness information with respect to image height at 501 is constant as in FIG. 5D, and in which shading (a state in which a region surrounding a central portion is darker than the central portion) does not occur. This is because, in the case where shading exists as in FIG. 5B, there is a difference in brightness with respect to image height in the first region α, which is to be extracted. Here, supposing that central portions are made to match in terms of brightness by gradation changing, the central portions are well made to match in terms of brightness with respect to image height (FIG. 5C); however, a diagnosis-possible region is a limited region in the central portions. Thus, in the case of, for example, FIG. 5B, since a diagnosis area is limited, the first region α and the second region β need to be regions that correspond to each other. However, when the brightness with respect to image height is constant as in FIG. 5D, since the brightness of the first region α is constant at any image height, the second region β does not have to be at a position that corresponds to the first region α. In addition, in the case of, for example, FIG. 5E acquired by performing gradation changing for FIG. 5D, the image regions are entirely well matched also in the surrounding portion in terms of brightness, and thus the entirety of each of the image regions is a diagnosis-possible region.

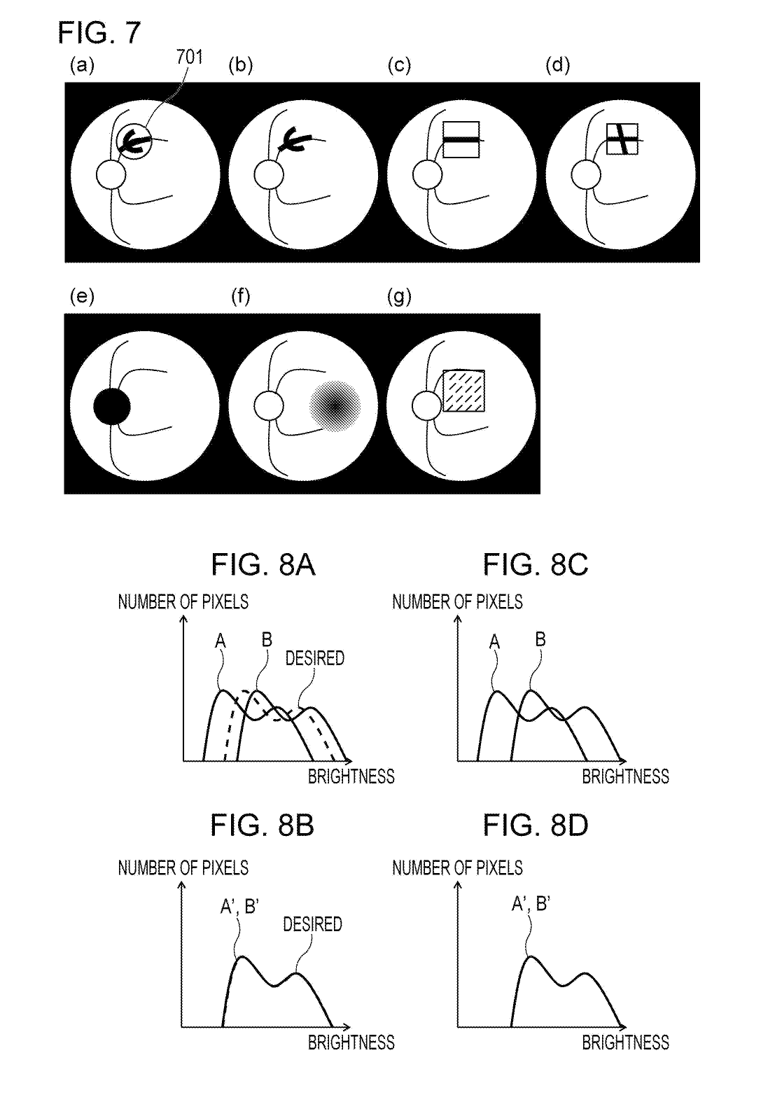

Here, although the first region α is desirably a blood vessel portion, the first region α may also be extraction examples (a) to (g) illustrated in FIG. 7. (a) illustrates an example in which an examiner specifies an image region 701 including a blood vessel portion corresponding to a blood vessel of the fundus image Er′, which has been captured, and the first region extraction unit 402, which extracts the first region α, extracts the blood vessel portion corresponding to the blood vessel of the fundus image Er′, which has been captured, from the specified image region 701. As a result, an effect may be expected in that the degree of freedom of analysis is increased by allowing a user to freely select the first region α. (b) illustrates an example in which the first region extraction unit 402 automatically extracts the blood vessel portion corresponding to the blood vessel of the fundus image Er′, which has been captured. As a result, a user does not have to select the first region α, an analysis time may be shortened, and a diagnosis support effect may be expected when the user is an unskilled person.

In addition, the form of a blood vessel to be selected may be, for example, as in (c) or (d). (c) illustrates an example in which a blood vessel to be extracted does not have a crossing portion and is a single blood vessel portion. This illustrates an example in which the first region α to be selected may be extracted if a region corresponds to a blood vessel. In addition, (d) illustrates an example in which blood vessels to be extracted cross with each other. This may be easily detected in the case of automatic extraction, and an effect may be expected in that a user easily selects an identical portion even in the case of manual selection and extraction. In addition, as in (e) to (g), an optic disk portion (e) corresponding to the optic disk of the fundus image Er′, which has been captured, a macula portion (f) corresponding to a macula, and a normal nerve fiber layer portion (g) corresponding to a normal nerve fiber layer may also be extracted. In many cases, (e) and (f) are circular and are characteristic portions in a fundus image. Thus, an effect may be expected in that a user easily makes a selection, and, for (g), an effect may be expected in that a region to be extracted may be freely selected.

Next, the image gradation changing unit 403, which is an example of the changing means, calculates, using coefficient calculation means, a coefficient with which the first region α and the second region β are to have a desired brightness, and performs gradation changing on at least one of the gradation of the first image A and the gradation of the second image B using the coefficient (S303). FIGS. 8A to 8D illustrate examples of a histogram in the case where gradation changing has been performed. For a histogram of FIG. 8A, an example is illustrated in which the image gradation changing unit 403 performs gradation changing on the first image A and the second image B such that the first region α and the second region β have a desired brightness, and a histogram of FIG. 8B is acquired. For a histogram of FIG. 8C, an example is illustrated in which the image gradation changing unit 403 performs gradation changing on the first image A such that the first region α has the same brightness as the second region β, and a histogram of FIG. 8D is acquired. Next, a display controller, which is not illustrated, causes the display 23 to display an image A′ and an image B′, the image A′ being acquired by performing gradation changing on the first image A, the image B′ being acquired by performing gradation changing on the second image B, examples of the display 23 including a display and a monitor. Here, in the case where the first region α has been extracted, the display controller causes the display 23 to perform display such that a display form illustrating the second region β is superimposed on the second image B. In the end, the image gradation changing ending unit 404 ends image gradation changing (S304).

Note that in addition to the above-described gradation changing, shading correction may also be performed on the first image A and the second image B. Note that FIGS. 6A and 6B illustrate diagrams for shading correction using the pupil diameter of the eye E to be inspected. Generally, it is known that in the case where an image of the fundus Er of the eye E to be inspected is captured, when the pupil diameter of the eye E to be inspected is small, shading occurs in a peripheral portion of the image due to vignetting of luminous flux. Image brightness with respect to image height of an image in which shading has occurred is denoted by 602 in contrast to 603 in FIG. 6A, and brightness decreases at a portion having a higher image height than the central portion. Thus, for example, when an anterior ocular segment is observed, a correction unit, which is not illustrated, may extract the pupil diameter (601) from an anterior ocular segment image of the eye E to be inspected and calculate shading expected on the basis of the extracted pupil diameter, and may also perform correction.

### Second Embodiment

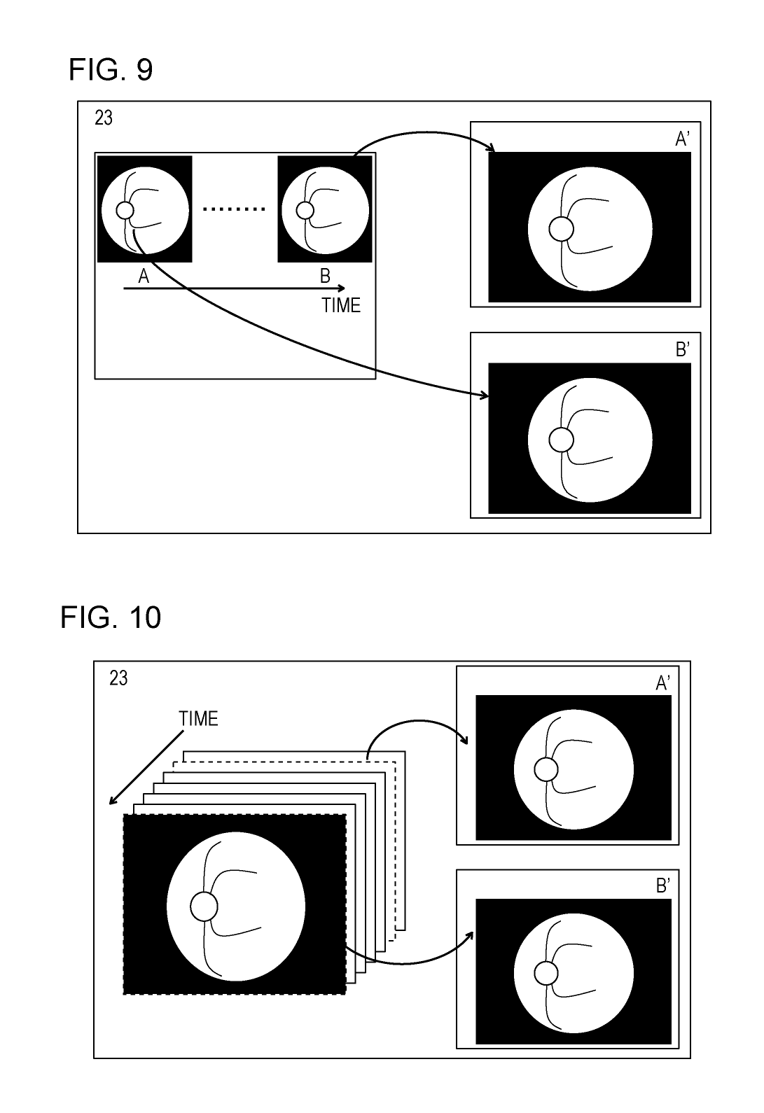

Next, a second embodiment will be described in detail using FIGS. 9 to 14. The present embodiment relates to a method for selecting and a method for displaying the first image A and the second image B. Note that a program (flow) of the present embodiment partially differs from that of the ophthalmic imaging apparatus of the first embodiment; however, an ophthalmic imaging apparatus of the present embodiment has the same configuration as that of the first embodiment.

Image Selection Screen

First, in FIG. 9, in a method for selecting an image on which gradation changing is to be performed, images stored in memory means are arranged horizontally on an imaging date basis and the first image A and the second image B may be selected from among the images using a mouse, a touch panel, or the like. There may also be a third image C and a fourth image D as images to be selected here. This has an effect in that the number of pieces of data may be increased in the case where there are a plurality of images to be compared with or when a change over time is checked. In addition, in the method for selecting, a plurality of images may be temporally arranged in a depth direction, and the first image A, which has been selected from among the plurality of images, may be displayed in such a manner that, for example, the first image A is zoomed and displayed on the right side as in FIG. 10. Here, FIG. 10 illustrates images arranged in an anterior-posterior direction on the imaging date basis, and a selection may be made from among the images. This has an advantage in that an image may be displayed in a zoomed manner even in the case where there are many captured images and in that images whose imaging dates differ greatly may be easily selected.

Gradation-Processed Image Display Flow

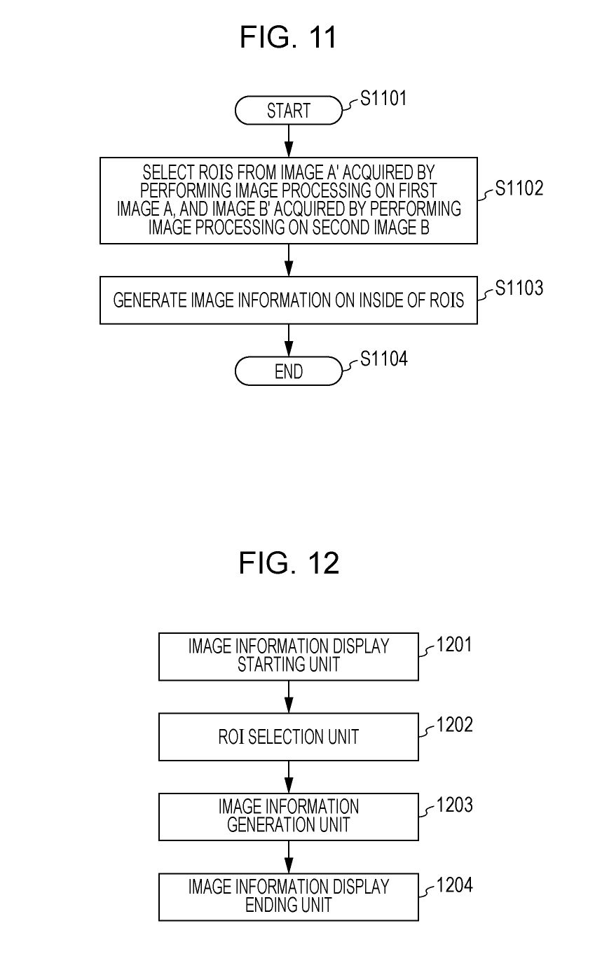

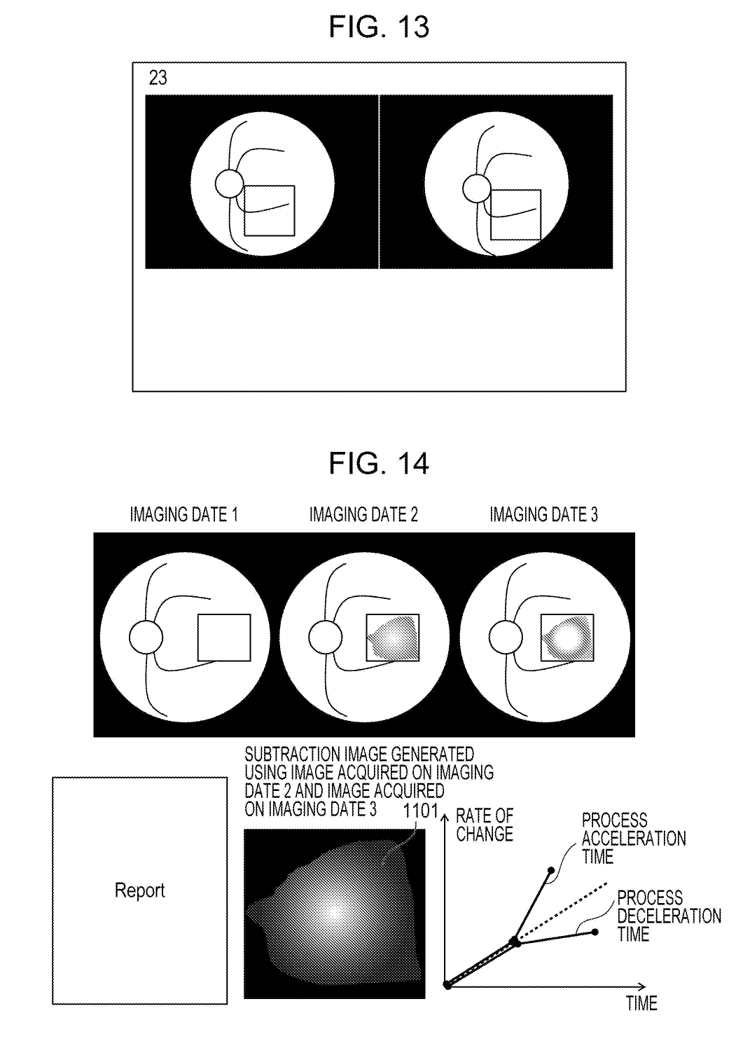

FIGS. 11 and 12 are a flowchart and a block diagram for displaying the image A′ and the image B′ acquired by performing, on the first image A and the second image B, image processing almost the same as that performed by the ophthalmic imaging apparatus described in the first embodiment. First, an image information display starting unit 1201 starts image information display (S1101). Next, an ROI selection unit 1202 selects ROIs in the image A′ and the image B′ displayed on the display 23 (S1102). Here, as in FIG. 13, when a user specifies one of the ROIs, for the other ROI, a ROI corresponding to the specified ROI is selected in conjunction with specification. Next, an image information generation unit 1203 generates image information on the inside of the ROIs (S1103). For example, a color-map generation unit, which is not illustrated, generates a color map in which coloring is performed for each brightness inside the specified ROIs. As a result, slight changes inside the ROIs may be made easier to see.

The display controller, which is not illustrated, causes the display 23 to display the image information on the inside of the ROIs, examples of the display 23 including a display and a monitor. Here, a display example is illustrated in FIG. 14. In the display example, report information is displayed in which the above-described image information is collected. Specifically, information acquired at the time of image changing is displayed such as a patient ID (Patient ID), gender, a disease name, a day of analysis (Date), the position and brightness value of each of the first region α and the second region β extracted by the extraction means, and a coefficient calculated by the coefficient calculation mean. As a result, information for facilitating performance of similar image processing next time may be provided in addition to information usually used by a doctor to conduct a diagnosis.

In addition, an imaging date of an image, which has been captured, is displayed at a position above the image on the display 23. That is, the display controller causes a plurality of fundus images to be displayed in time series in a three-dimensional manner (arranged and displayed along a time axis) in accordance with imaging dates of first and second fundus images acquired by performing autofluorescence imaging on the fundus at different times. In addition, as an example for illustrating a change in a disease portion in time series, using the brightness of a blood vessel portion after gradation changing as a basis, a line graph may also be displayed illustrating changes in the following regions in time series with respect to old information in images for which an ROI has been selected. The regions are specified, for example, as a hyperfluorescence region, which is a high-brightness region of the blood vessel portion, a low fluorescence region, which is a low-brightness region of the blood vessel portion, and a normal fluorescence region, which is an intermediate-brightness region. In the end, an image information display ending unit 1204 ends image information display (S1104).

Ophthalmic Imaging Apparatus According to Another Present Embodiment

Note that an ophthalmic imaging apparatus according to the other present embodiment may correct a brightness difference between images by performing gradation changing to acquire a desired brightness using image information of the first region, the brightness difference between images having been generated because of imaging conditions. In addition, as a result of changing of gradation from the brightness of the first region to the desired brightness, it is estimated using, as a basis, the brightness of a blood vessel portion, which does not show autofluorescence, that, for example, a brightness region having a brightness higher than the brightness of the blood vessel portion is a hyperfluorescence region, a region having a brightness lower than or equal to the brightness of the blood vessel portion is a low fluorescence region, and a region having an intermediate brightness is a normal fluorescence region in an image acquired after changing. In addition, this ophthalmic imaging apparatus makes it possible to draw a change in a disease region and determine the form of a disease portion by performing color display in accordance with a brightness gradation. In addition, the first region may be extracted either manually or automatically in the ophthalmic imaging apparatus according to the other present embodiment. In the case of manual extraction, there are advantages in that a user may specify a desired image region as the first region and the degree of freedom of analysis is increased. These are especially effective when the user is a skilled person or the like. In contrast, in the case of automatic extraction, labor may be reduced, and also automatic extraction is especially effective as a diagnosis support tool when the user is an unskilled person or the like. In addition, information on a disease portion in which a user is interested may be displayed by selecting an ROI (Region of Interest: a region of interest). Thus, a time needed to conduct a diagnosis may be shortened. In addition, the ophthalmic imaging apparatus according to the other present embodiment may align a plurality of images of the same fundus such that the positions of characteristic points in the images match, specify a first region and an ROI of one image, and specify a second region, which corresponds to the first region, and an ROI of another image on the basis of the first region and a result of image alignment. As a result, a complicated operation may be prevented in which the first region is specified in the plurality of images, and labor involved in the operation may be reduced. Furthermore, the ophthalmic imaging apparatus according to the other present embodiment may specify ROIs that correspond to each other in a plurality of gradation corrected images of the same eye, and may calculate, for each ROI, the number of pixels of a hyperfluorescence region and the number of pixels of a low fluorescence region and display the numbers of pixels as time-series change information. Thus, it is possible to effectively support determination of changes in time series in the ROI, and the ophthalmic imaging apparatus according to the other present embodiment is especially effective as a diagnosis support tool.

### Other Embodiments

In addition, the present invention is also realized by executing the following process. That is, the following process is a process in which a software program (program) that realizes functions of the above-described embodiments is supplied to a system or an apparatus via a network or various recording medium and the program is read and executed by a computer of the system or of the apparatus (or a CPU, an MPU, or the like).

According to the present invention, in a plurality of fundus images acquired at different times by performing autofluorescence imaging on the fundus of an eye to be inspected, for example, gradations of the plurality of fundus images may be matched using regions which are relatively less likely to be affected by changes in lipofuscin that occur over time such as blood vessels. As a result, in a plurality of fundus images acquired at different times by performing autofluorescence imaging on the fundus of the eye to be inspected, for example, gradations of regions may be matched which are less likely to be affected by changes in lipofuscin that occur over time. Thus, false diagnoses may be reduced in which a user diagnoses a region which is less likely to be affected by changes in lipofuscin in a fundus image as a region where a change in lipofuscin occurs.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
determination means that determines a first region and a second region, the first region being a portion of a first fundus image acquired by performing autofluorescence imaging on a fundus of an eye to be inspected, the second region being a portion of a second fundus image acquired by performing autofluorescence imaging on the fundus at a time different from that of the first fundus image and being at a position corresponding to the first region; and
changing means that changes a gradation of at least one of the first and second fundus images on the basis of pixel values of the first and second regions.

2. The image processing apparatus according to claim 1, wherein the determination means determines, as the first and second regions, regions which are less likely to be affected by a change in lipofuscin that occurs over time in the first and second fundus images.

3. The image processing apparatus according to claim 1, wherein the determination means determines, as the first and second regions, regions which include a blood vessel of the fundus in the first and second fundus images.

4. The image processing apparatus according to claim 1, wherein the changing means changes the gradation of at least one of the first and second fundus images such that pixel values of the first region become almost equal to pixel values of the second region.

5. The image processing apparatus according to claim 1, wherein the changing means changes a gradation of the first fundus image such that pixel values of the first region become equal to pixel values of the second region, or changes gradations of the first and second fundus images such that pixel values of the first and second regions become equal to certain pixel values.

6. The image processing apparatus according to claim 1, further comprising:
coefficient acquisition means that acquires a coefficient with which pixel values of at least one of the first and second fundus images are to be corrected on the basis of pixel values of the first and second regions; and
correction means that corrects pixel values of at least one of the first and second fundus images using the coefficient, wherein
the changing means changes the gradation of at least one of first and second fundus images on the basis of the corrected pixel values.

7. The image processing apparatus according to claim 1, further comprising:
display control means that causes, in a case where the first region has been determined by the determination means, display means to perform display such that a display form illustrating the second region is superimposed on the second fundus image.

8. The image processing apparatus according to claim 7, wherein the display control means causes the first and second fundus images to be arranged and displayed along a time axis.

9. The image processing apparatus according to claim 7, wherein the display control means causes the first and second fundus images to be displayed in time series in a three-dimensional manner in accordance with imaging dates of the first and second fundus images.

10. The image processing apparatus according to claim 1, further comprising:
selection means that selects the first and second fundus images from among a plurality of fundus images acquired by performing autofluorescence imaging on the fundus; and
alignment means that aligns the first and second fundus images using certain regions of the first and second fundus images, wherein
the changing means changes the gradation of at least one of the first and second fundus images that have been aligned.

11. The image processing apparatus according to claim 1, further comprising:
correction means that corrects shading of the first and second fundus images on the basis of a pupil diameter of an anterior ocular segment image of the eye to be inspected.

12. An image processing apparatus comprising:
determination means that determines a first region and a second region, the first region being a portion of a first image acquired by performing imaging on an object to be inspected, the second region being a portion of a second image acquired by performing imaging on the object to be inspected at a time different from that of the first image and being at a position corresponding to the first region; and
changing means that changes a feature value of at least one of the first and second images on the basis of feature values of the first and second regions.

13. The image processing apparatus according to claim 12, further comprising:
determination means that determines a gradation changing characteristic on the basis of brightness distributions, which are the feature values of the first and second regions, wherein
the changing means changes a brightness distribution of at least one of the first and second images using the determined gradation changing characteristic.

14. The image processing apparatus according to claim 12, wherein the determination means determines, as the first and second regions, regions which are less likely to be affected by a change in lipofuscin that occurs over time in the first and second images.

15. The image processing apparatus according to claim 12, wherein the determination means determines, as the first and second regions, regions which include a blood vessel of the object to be inspected in the first and second images.

16. The image processing apparatus according to claim 1 in an ophthalmic system in which the image processing apparatus is connected to an ophthalmic imaging apparatus that performs autofluorescence imaging on the fundus of the eye to be inspected such that communication is possible, the image processing apparatus further comprising:
acquisition means that acquires fundus image data transmitted from the ophthalmic imaging apparatus.

17. An ophthalmic imaging apparatus comprising:
an illumination optical system that illuminates an eye to be inspected;
an imaging optical system that captures an image of a fundus of the eye to be inspected, on the basis of light returned from the eye to be inspected which is illuminated by the illumination optical system;
selection means that selects an autofluorescence imaging mode in which autofluorescence imaging is performed on the fundus;
wavelength selection means that is inserted, in a case where the autofluorescence imaging mode has been selected, into at least one of the illumination optical system and the imaging optical system;
determination means that determines a first region and a second region, the first region being a portion of a first fundus image acquired by performing autofluorescence imaging on the fundus using the imaging optical system, the second region being a portion of a second fundus image acquired by performing autofluorescence imaging on the fundus using the imaging optical system at a time different from that of the first fundus image and being at a position corresponding to the first region; and
changing means that changes a gradation of at least one of the first and second fundus images on the basis of pixel values of the first and second regions.

18. The ophthalmic imaging apparatus according to claim 17, wherein
the wavelength selection means includes
an autofluorescence exciter filter, which is insertable into and removable from an optical path of the illumination optical system, and
an autofluorescence barrier filter, which is insertable into and removable from an optical path of the imaging optical system.

19. The ophthalmic imaging apparatus according to claim 17, wherein the determination means determines, as the first and second regions, regions which are less likely to be affected by a change in lipofuscin that occurs over time in the first and second fundus images.

20. The image processing apparatus according to claim 17, wherein the determination means determines, as the first and second regions, regions which include a blood vessel of the fundus in the first and second fundus images.

21. An image processing method comprising:
a step of determining a first region and a second region, the first region being a portion of a first fundus image acquired by performing autofluorescence imaging on a fundus of an eye to be inspected, the second region being a portion of a second fundus image acquired by performing autofluorescence imaging on the fundus at a time different from that of the first fundus image and being at a position corresponding to the first region; and
a step of changing a gradation of at least one of the first and second fundus images on the basis of pixel values of the first and second regions.

22. An image processing method comprising:
a step of determining a first region and a second region, the first region being a portion of a first image acquired by performing imaging on an object to be inspected, the second region being a portion of a second image acquired by performing imaging on the object to be inspected at a time different from that of the first image and being at a position corresponding to the first region; and
a step of changing a feature value of at least one of the first and second images on the basis of feature values of the first and second regions.

23. The image processing method according to claim 22, further comprising:
a step of determining a gradation changing characteristic on the basis of brightness distributions, which are the feature values of the first and second regions, wherein
in the step of changing, a brightness distribution of at least one of the first and second images is changed using the determined gradation changing characteristic.

24. The image processing method according to claim 21, wherein in the step of determining, regions which are less likely to be affected by a change in lipofuscin that occurs over time in the first and second fundus images are determined as the first and second regions.

25. The image processing method according to claim 21, wherein in the step of determining, regions which include a blood vessel of the fundus in the first and second fundus images are determined as the first and second regions.

26. A program that causes a computer to execute steps of the image processing method according to claim 21.
